(19) United States
(12) Patent Application Publication
(10) Pub. No.: US 2025/0018143 A1
(43) Pub. Date: Jan. 16, 2025

(54) A SEQUENTIAL, MULTI-MODAL, MULTI-PATIENT MECHANICAL VENTILATION SYSTEM

(71) Applicant: JOMOCO, INC., Bethesda, MD (US)

(72) Inventors: Jack A. RAGHEB, North Bethesda, MD (US); Christopher TKACH, Columbus, OH (US); Shafiq Ahmad CHAUDHURI, Great Falls, VA (US)

(21) Appl. No.: 18/709,860

(22) PCT Filed: Nov. 17, 2022
(86) PCT No.: PCT/US2022/050184

§ 371 Date: May 14, 2024

Related U.S. Application Data
(60) Provisional application No. 63/264,192, filed on Nov. 17, 2021.

(51) Int. Cl. A61M 16/20; A61M 16/00; A61M 16/10; A61M 16/12; B33Y 10/00; B33Y 80/00

(52) U.S. Cl. CPC A61M 16/203; A61M 16/0051; A61M 16/1005; A61M 16/12; A61M 16/208; B33Y 10/00; B33Y 80/00; A61M 2016/0027; A61M 2016/0033; A61M 2209/10

(57) ABSTRACT

A multifunctional ventilation device is provided herein. The multifunctional ventilation device includes an intake manifold assembly configured to receive gas from one of a plurality of gas sources. The multifunctional ventilation device also includes a first and second outlet manifold assembly being configured to deliver continuous, periodic, sequential or simultaneous airflow. The multifunctional ventilation device also includes a first and second proportional master air valve configured to provide precise airflow, the first and second proportional master air valve being in fluidic connection with at least the first and second outlet manifold assemblies, respectively. The multifunctional ventilation device also includes a control system programmed to selectively provide continuous, periodic, sequential or simultaneous delivery of the gas to a patient air delivery system. Each of the first and second plurality of patient outlets, of the multifunctional ventilation device, are fluidically equidistant from the first and second proportional master air valves, respectively.

### CROSS-REFERENCE TO RELATED APPLICATIONS

The present application claims priority under 35 U.S.C. § 119 (e) to U.S. Provisional Patent Application No. 63/264,192, filed Nov. 17, 2021, which is incorporated herein by reference in its entirety.

### BACKGROUND

There are a range of indications for invasive mechanical ventilation (IMV) including increasing respiratory rate, asynchronous respiratory pattern, a change in mentation and level of consciousness, frequent oxygen desaturation despite increasing oxygen concentration, hypercapnia and respiratory acidosis, circulatory problems, including hypotension and atrial dysrhythmias. As an example, roughly 5% of patients hospitalized with COVID-19 are admitted to the Intensive Care Unit (ICU) and 70-90% of those admitted require IMV. The COVID-19 pandemic exposed a global shortage of mechanical ventilators. Resource-poor areas are especially vulnerable to demand spikes and locations with high morbidity have struggled to outfit hospitals with needed equipment.

IMV, which may require the patient to be rendered unconscious, is only considered when a patient's spontaneous breathing is unable to adequately exhaust carbon dioxide or oxygenate blood, both critical medical states, the latter of which is termed hypoxemia. Normally, breathing is accomplished by the creation of negative pressure within the chest cavity, which draws air into the airways, inflating the lungs; enabling gas exchange between the small air sacs within the lung (pulmonary alveoli) and capillary blood vessels. Whereas inhalation is an active process, ordinarily exhalation and lung deflation occur passively. While IMV also relies on passive exhalation, it delivers positive pressure during inspiration (PIP) to inflate the lungs and positive end expiratory pressure (PEEP) to prevent the collapse of alveoli at the end of exhalation.

Acute respiratory distress syndrome (ARDS), a complication of COVID-19, is the result of an abnormal increase in pulmonary capillary permeability, resulting in fluid accumulation in the lungs (pulmonary edema). Other causes of ARDS include pneumonia or severe flu, sepsis, a severe chest injury accidentally inhaling vomit, smoke or toxic chemicals, near drowning, acute pancreatitis, and an adverse reaction to a blood transfusion. Consequences include reduced compliance of pulmonary tissue and decreased oxygen diffusion, increasing the work of breathing and causing hypoxemia. The lungs of a patient with ARDS will often fail to inflate fully, resulting in regions of collapsed alveoli, further reducing alveolar surface area and worsening hypoxemia. IMV can overcome these impediments to ventilation through several means. Sufficiently high PIP overcomes the increased resistance to ventilation due to reduced lung compliance, reducing the patient's work of breathing, and improving gas exchange. Additionally, the use of PEEP, in which a residual amount of positive pressure is maintained in the lungs at the end of exhalation, reduces alveolar collapse, thus maintaining alveolar surface area and improving oxygenation. Another means of overcoming hypoxemia is to increase the concentration of oxygen delivered to the lungs, a parameter known as fraction of inspired oxygen (FiO₂), which promotes alveolar O₂ exchange. While all conventional mechanical ventilators have these essential features, a multi-patient ventilator that incorporates these features and can individualize these outputs to each patient based on their individual need for PIP and FiO₂ has not been described. Multi-patient ventilators have been described previously. Many involve a bag that self-fills with air, mounted onto a frame, situated such that a motor-powered arm applies compressions at set intervals. Other models use a pneumatic based or blower (fan) based ventilation blower design. A small number have focused on radically minimalist, easily manufactured designs. These designs have several major limitations. Bag compression solutions greatly increase the risk of nosocomial infection and ventilator-associated lung injury (VALI). Motorized air-bag compression systems, while relatively cheap, are not mechanically suited for long-term ventilation. Small, inexpensive motors running for long periods often run into electrical/mechanical problems such as overheating and wear/tear, especially when there are many moving components (motor, gear train, cam-arm). Minimalist designs have fewer moving parts and are thus easy to 3D-print, but are not suited for long-term ventilation, require frequent calibration, and are difficult to operate. Overall, a common drawback of low-cost designs is the lack of easily programmable inputs.

Thus, it would be desirable to provide an improved multifunctional ventilation device.

### SUMMARY

One aspect of the invention provides a multifunctional ventilation device. The multifunctional ventilation device includes an intake manifold assembly configured to receive gas from at least one of a plurality of gas sources. The multifunctional ventilation device also includes a first outlet manifold assembly configured and adapted to be in fluidic connection with the intake manifold assembly, the first outlet manifold assembly including a first plurality of patient outlets, the first outlet manifold assembly being configured and adapted to deliver continuous airflow. The multifunctional ventilation device also includes a second outlet manifold assembly configured and adapted to be in fluidic connection with the intake manifold assembly, the second outlet manifold assembly including a second plurality of patient outlets, the second outlet manifold assembly being configured and adapted to deliver periodic, sequential or simultaneous airflow. The multifunctional ventilation device also includes a first proportional master air valve configured to provide precise airflow to the first outlet manifold assembly, the first proportional master air valve being in fluidic connection with at least the first outlet manifold assembly. The multifunctional ventilation device also includes a second proportional master air valve configured to provide precise airflow to the second outlet manifold assembly, the second proportional master air valve being in fluidic connection with at least the second outlet manifold assembly.

The multifunctional ventilation device also includes a control system. The control system includes a plurality of valves including the first and second proportional master air valve. The control system also includes a plurality of sensors. The control system also includes a processor configured to control the plurality of valves, and measure data from the plurality of sensors. The control system is programmed to selectively provide continuous, periodic, sequential or simultaneous delivery of the gas to a patient air delivery system.

Each of the first plurality of patient outlets, of the multifunctional ventilation device, are fluidically equidistant from the first proportional master air valve. Each of the second plurality of patient outlets, of the multifunctional ventilation device, are fluidically equidistant from the second proportional master air valve.

### Definitions

The instant invention is most clearly understood with reference to the following definitions.

As used herein, the singular form “a,” “an,” and “the” include plural references unless the context clearly dictates otherwise.

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

As used in the specification and claims, the terms “comprises,” “comprising,” “containing,” “having,” and the like can have the meaning ascribed to them in U.S. patent law and can mean “includes,” “including,” and the like.

Unless specifically stated or obvious from context, the term “or,” as used herein, is understood to be inclusive.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (as well as fractions thereof unless the context clearly dictates otherwise).

### BRIEF DESCRIPTION OF THE DRAWINGS

For a fuller understanding of the nature and desired objects of the present invention, reference is made to the following detailed description taken in conjunction with the accompanying drawing figures wherein like reference characters denote corresponding parts throughout the several views.

FIG. 1 illustrates the upper manifold and valve arrangement for Continuous Positive Airway Pressure (CPAP) and PEEP Emulation in P-, M- and S-Modes of operation, in accordance with an exemplary embodiment of the present disclosure.

FIGS. 2A-2B illustrate the lower manifold and valve arrangement for M- and S-Modes of operation, in accordance with an exemplary embodiment of the present disclosure.

FIG. 3 illustrates the lower manifold and valve arrangement for the P-Mode of operation, in accordance with an exemplary embodiment of the present disclosure.

FIG. 4 illustrates an oblique frontal view of upper (CPAP/PEEP) and lower (Splitter/Ventilator) subunits, in accordance with an exemplary embodiment of the present disclosure.

FIG. 5 illustrates an oblique rear view of the upper (CPAP/PEEP) and lower (Splitter/Ventilator) subunits, in accordance with an exemplary embodiment of the present disclosure.

FIG. 6 illustrates a schematic view of inputs and outputs of an exemplary embodiment of the present disclosure.

FIGS. 7A-7B illustrates a housing, in accordance with an exemplary embodiment of the present disclosure.

### DETAILED DESCRIPTION OF THE INVENTION

The present disclosure describes a multifunctional ventilation device. In certain exemplary embodiments, the device is configured to simultaneously support multiple (e.g., 2, 3, 4, greater than 4, etc.) patients by interfacing with an existing ventilator to either serve as a conventional splitter (“S-Mode”), delivering the same ventilator output to each patient, or as a multiplier (“M-Mode”) that can sequentially provide PIP and FiO₂ individualized to the needs of each patient. In certain embodiments, additionally, the device can function as a standalone pneumatic IMV (“P-Mode”) that can sequentially provide PIP and FiO₂ individualized to the needs of each patient, or as a CPAP device with FiO₂. In certain embodiments, the device is configured to execute a self-cleaning function (e.g., “self-cleaning mode”).

When coupled to an existing ventilator (M-Mode) or a source of pressurized air and oxygen (P-Mode), the device delivers sequential ventilation (e.g., rather than simultaneous ventilation to multiple patients (e.g., 2, 3, 4, greater than 4, etc.)). In contrast to ventilator splitters, certain exemplary embodiments of the present disclosure enables integrated pressure and oxygen support individualized to the needs of each patient while precluding cross-contamination of airflow between patients. Such devices can also serve as a ventilator splitter (S-Mode) or a stand-alone CPAP device, delivering the same output to each patient. Certain exemplary embodiments utilize electronic circuitry to achieve fully integrated control of ventilation parameters.

Certain exemplary embodiments can be configured to operate as an IMV or a non-invasive CPAP, can be configured to connect with and blend multiple external gas sources (e.g., 2) as intake, and can be configured to interface with a separate ventilator unit as intake. Certain exemplary embodiments can provide individualized PIP and FiO₂ as well as adjustable PEEP through a second, continuous flow delivery manifold.

In M-Mode and P-Mode of an exemplary device of the present disclosure, ventilation of each patient can be staggered, enabling each patient to be ventilated independently of the other patients connected to the device. In M-Mode, pressurized air from an associated ventilator (oxygen is connected directly to the device), set to maximum PIP, can be fed into the device, which can act as a “multiplier” (e.g., a 4-way multiplier), enabling sequential, independent delivery of FiO₂ and PIP individualized to the needs of each patient. The maximum number of patients that can be independently ventilated is a function of the Inspiration time (I time), I:E Ratio (inspiratory/expiration time ratio) and Respiratory Rate (RR) (e.g., between 9-15 breaths per minute). The I:E ratio can start around 1:2 and then be adjusted using the electronic controller.

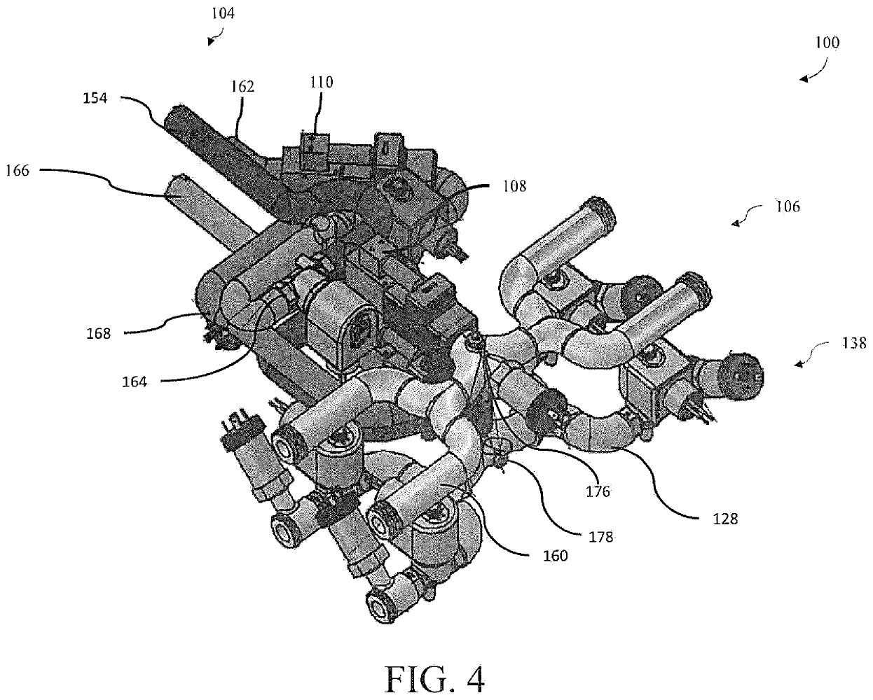

Based on the inputs entered into the associated ventilator, a microprocessor of the device can be configured to calculate corresponding parameters for ease of use and to avoid operator error. The device can be configured to emulate positive-end expiratory pressure (PEEP) by delivering CPAP through a first outlet manifold assembly (depicted as the upper manifold assembly in FIG. 4) independent of that which delivers PIP. In M-Mode and P-Mode, the device can be configured such that I time, RR and PEEP can be adjustable (and the same) for each patient. In P-Mode, the device can deliver PIP using a source of pressurized air rather than an existing ventilator; RR and I time can be controlled through the device. Otherwise, the P-Mode and M-Mode do not functionally differ. Because of the relationship between RR and I time, in P-Mode, a broader range of RR and I time is achievable for a given number of patients as compared to M-Mode. A one-way valve can be used to prevent expired air from contaminating an outflow track of the device and/or other patients. Each patient's exhalation, which poses a nosocomial infection risk, can be independently filtered (e.g., to prevent aerosolization). Sensors can be positioned to measure any potential pressure irregularities (e.g., due to leaks, valve failure, airway obstruction, changes in lung resistance, etc.). If such irregularities are detected, such detection can trigger visual and/or auditory alerts that manifest (e.g., appear) on or near the user interface.

In certain embodiments of the present disclosure, a multifunctional ventilation device includes a branching manifold and sequential delivery of a pressurized ventilator gas to patients. Certain embodiments of the present disclosure requires only a single regulatory pressure valve to control the pressure of a gas delivered to a plurality (e.g., four) of branches and each branch utilizes one on-off valve downstream (rather than upstream of the single regulatory pressure valve shared in common by the branches). Certain embodiments of the present disclosure operate in a sequential fashion in order to allow individualized control of the positive inspiratory pressure (PIP) and fraction of inspired oxygen (FiO₂) delivered to each patient based on their specific needs. Certain embodiments of the present disclosure employ two separate manifolds, one to deliver sequential invasive ventilation and the other to deliver Continuous Positive Airway Pressure (CPAP) ventilation. These manifolds may operate either in isolation or in parallel, the later operation being a means of emulating PEEP during invasive ventilation. Certain embodiments of the present disclosure employ two separate gas intakes. Within the intake manifold assembly, gases (e.g., oxygen, nitrogen, air, etc.) are blended by means of a gas lens to a specific concentration (e.g., FiO₂) individualized to the needs of each patient by utilizing a single regulatory valve shared in common by the branches (e.g., 4 branches) of the manifold. Certain embodiments of the present disclosure can also deliver a specified FiO₂ when providing CPAP.

Additionally, certain embodiments of the present disclosure can also function in combination with a conventional ventilator to deliver either constant pressure or constant volume ventilation to multiple patients. Certain embodiments of the present disclosure include operations that require regulatory control valves and software programming.

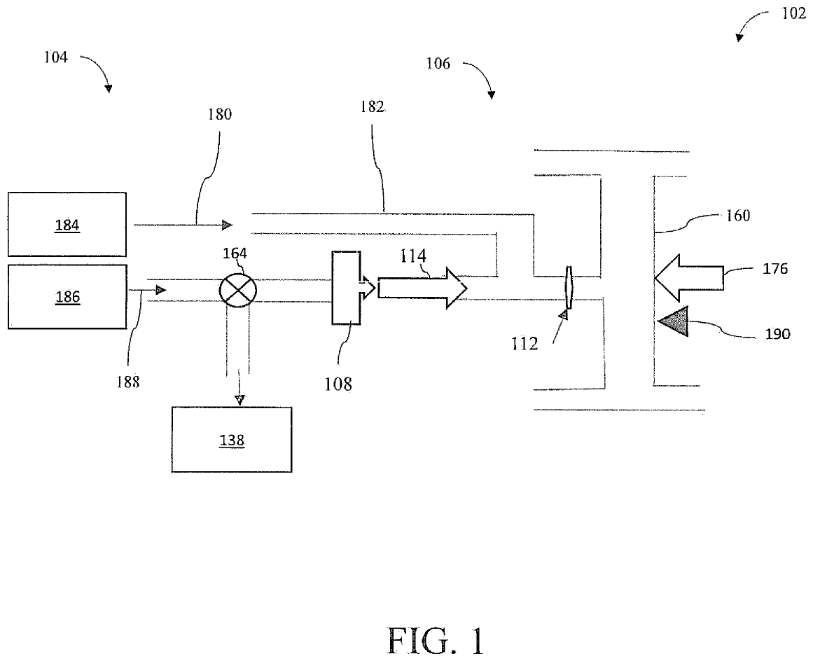

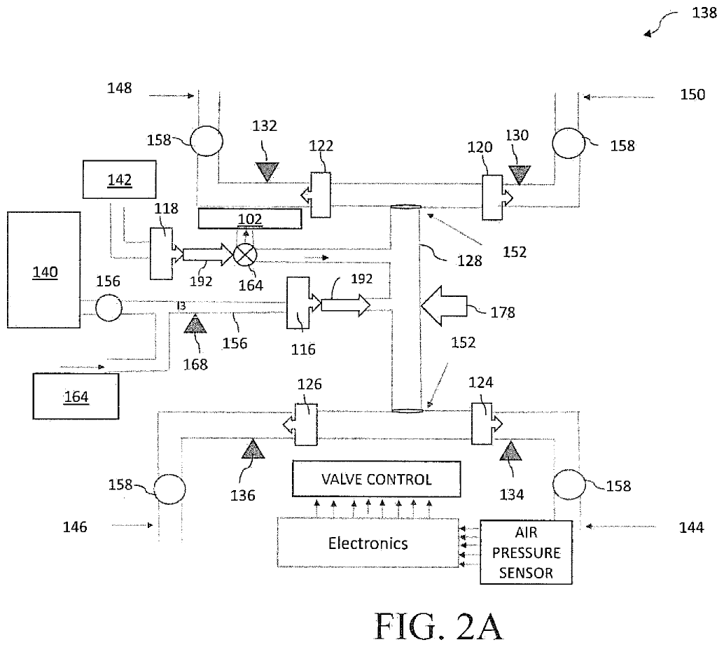

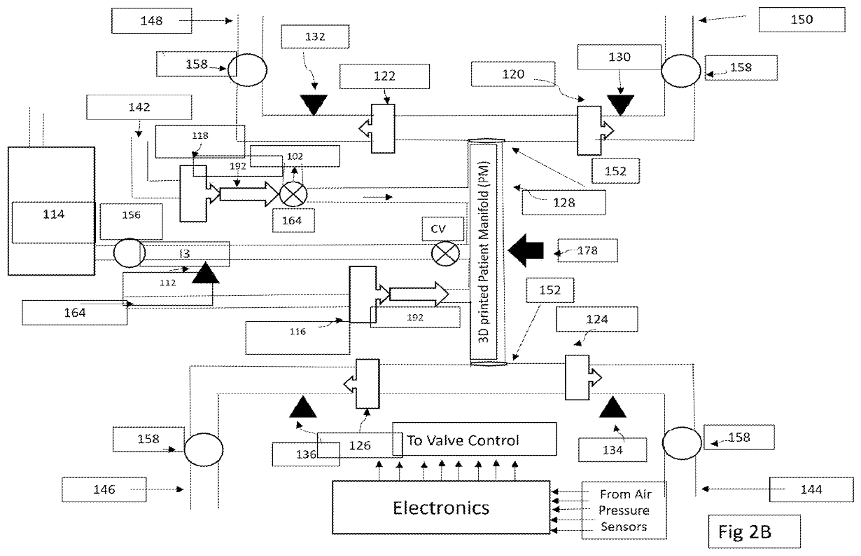

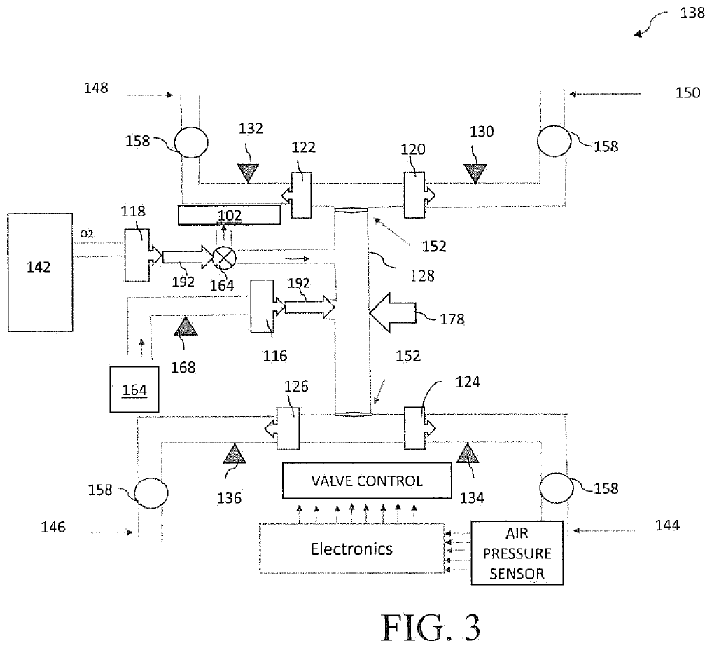

Embodiments of the present disclosure can be better described in connection with the drawings. Referring now to the drawings, FIGS. 1-3 illustrate functional modes schematically, in connection with certain exemplary embodiments of the present disclosure. Each figure depicts a static mechanical architecture of a dual manifold within a housing compartment, shown without electrical connections, wiring, or ingoing/outgoing conduits, in accordance with certain exemplary embodiments of the present disclosure. In FIG. 1, a first manifold assembly 102 is illustrated (which can be described as the “upper” manifold assembly). First manifold assembly 102 includes an intake manifold assembly 104 and an outlet manifold assembly 106. First manifold assembly 102 can be utilized in one or more (e.g., 1, 2, 3, 4, all, etc.) modes. In a CPAP Mode, the upper manifold assembly 102 can operate in isolation. In S-Mode, M-Mode, and P-Mode, the upper manifold assembly 102 can operate to emulate PEEP, delivered in conjunction with the PIP supplied via a second manifold assembly 108 (which can be described as the “lower” manifold assembly). The second manifold assembly 108 in M-Mode and S-Mode is shown in FIGS. 2A-2B. The same second manifold assembly 108 in P-Mode is shown in FIG. 3. FIGS. 2A-2B and FIG. 3 differ with respect to the source of pressurized air.

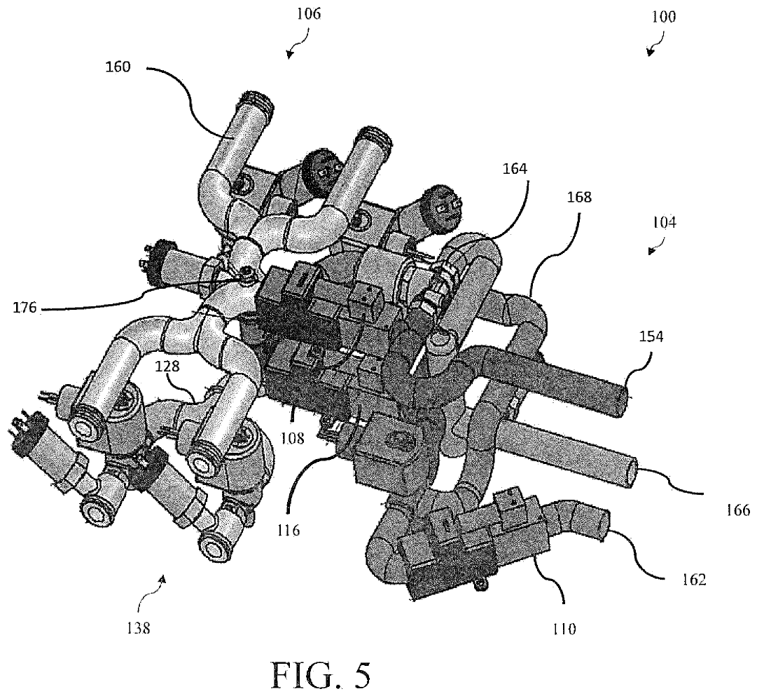

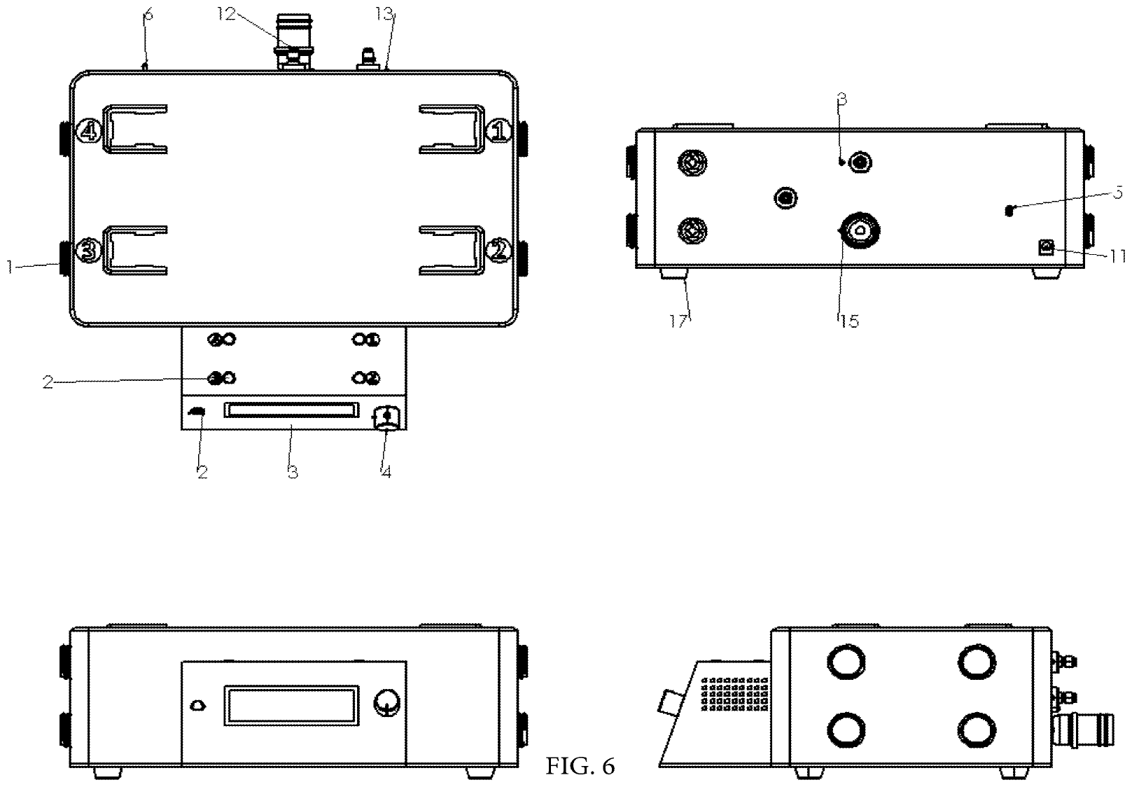

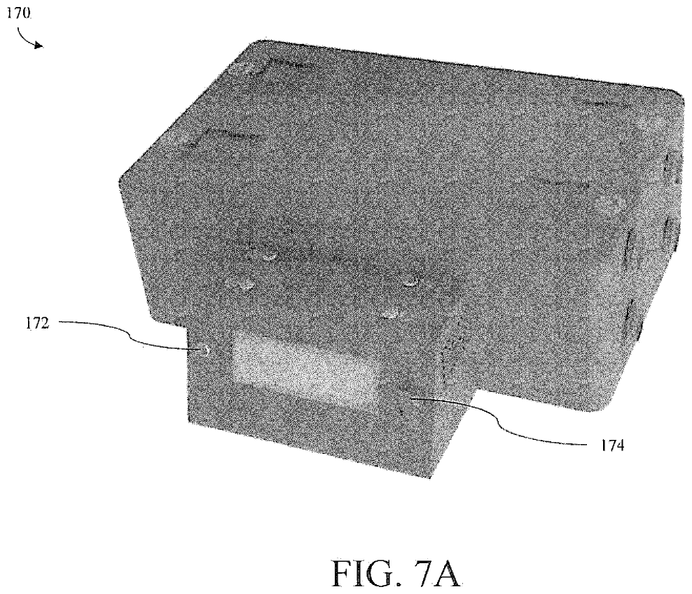

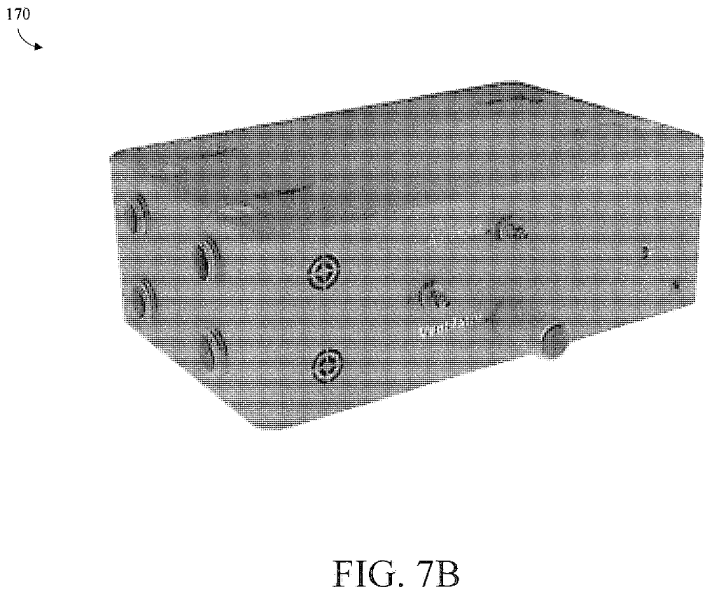

FIGS. 4 and 5 are CAD representations of a multifunctional ventilation device 100 (including assembled first manifold assembly 102 and second manifold assembly 108 within a housing unit), in accordance with exemplary embodiments of the present disclosure. FIG. 6 is a schematic of a housing compartment showing inputs and outputs. FIG. 7 is an illustration of an exterior housing compartment from several perspectives.

### Conceptualization

Certain embodiments of the present disclosure can be controlled through an Electronics Controller User Interface (ECUI), which also can display the sensor and alarm outputs. An ECUI 170 is illustrated in FIGS. 6 and 7. Once powered on, the operator can be prompted to enter a programming mode by depressing a program button 172. Once the mode of operation has been selected, all patient parameter values (e.g., RR, I time, FiO₂, PIP, PEEP, CPAP) can be entered using a rotary encoder 174. A given parameter can be selected by turning rotary encoder 174 clockwise (or counter clockwise). Following parameter selection, an operator can be prompted to confirm a selection by depressing the rotary 174 encoder once. The same sequence can be used to select a parameter value. A selection can be undone by depressing rotary encoder 174 twice.

In certain embodiments, ventilation parameters are input manually, by individually adjusting the requisite valves to produce parameter modifications.

### CPAP Mode

Certain exemplary embodiments can be configured to implement a “CPAP Mode” which enables such embodiments to act as a standalone CPAP device (e.g., using a hospital wall outlet or alternate gas source) to provide oxygenated pressurized air (e.g., 3-20 cm H₂0) to multiple patients. The CPAP mode may be best described in connection with FIG. 1, FIGS. 2A-2B, and FIG. 3. In FIG. 1, gas 180 (e.g., oxygen) is illustrated being transferred from oxygen transfer valve 184 to a manifold injector 182 (e.g., PEEP manifold oxygen injector). Gas 188 (e.g., air) is illustrated being provided from external/wall air source 186. Proportional mini valves 108 and 110, can be configured to regulate pressure and FiO₂, respectively (e.g., see FIG. 4). A gas lens 12 can help assure uniform mixing of pressurized air and oxygen. In certain embodiments, gas mixture can occur in the intake manifold assembly, the first outlet manifold assembly, or second outlet manifold assembly. In certain other embodiments, gas mixture (at least partially) can occur in an external mixture chamber. In certain embodiments, the driving gas is humidified, moisturized, or heated.

The flow sensor 114 can measure the output of the proportional mini valves 108 and 110 valves and can be used with a feedback control loop to adjust these valves to meet the programmed pressure and FiO₂ levels. The pressure level and FiO₂ can be programmed through the ECUI (e.g., ECUI of FIGS. 7A-7B). CPAP can be delivered through a manifold 160 (e.g., a 4-way manifold, a first outlet manifold, etc.) separate from a manifold that delivers oxygenated PIP. An identical configuration can be utilized to emulate PEEP in S-Mode, M-Mode, and P-Mode, except that in these modes the PEEP is not oxygenated (e.g., FiO₂=21%).

Certain methods of the present disclosure relate to implementing or using a CPAP mode. For example, one method includes the steps of: (a) electronically controlling (e.g., with precision) variably oxygenated Continuous Positive Airway Pressure delivered simultaneously to 1 or more patients; (b) using a gas lens to mix two gases to achieve a consistent gas mixture; (c) using a flow sensor to provide a feedback loop configured to regulate operation of a proportional air valve to assure delivery of the correct pressure.

### Splitter (S-) Mode—Non-Sequential Operation

The Splitter (S-) Modes (e.g., S-Volume Control (S-VC) and S-Pressure Control (S-PC)) allow a certain embodiments of the present disclosure to support multiple patients (e.g., four patients) by splitting the output of an accessory ventilator delivering either a fixed tidal volume (S-VC) or a fixed pressure (S-PC). Referring now to FIGS. 2A-2B (see also FIG. 4 and FIG. 5), an air pressure (flow) sensor 138 is illustrated. Air pressure sensor 138 can detect the pulsatile airflow from an accessory ventilator 140. Ventilation is delivered simultaneously to all active patient ports (in contrast to other modes, such as M-Mode and P-Mode), which receive the same ventilator output. As in M-Mode and P-Mode, PEEP can be emulated via the upper manifold assembly 102. In certain embodiments, all ventilator settings (except for PEEP) are entered into and controlled by the accessory ventilator 140.

Certain methods of the present disclosure relate to implementing or using a splitter mode. For example, one method includes the steps of: (a) electronically controlling (e.g., with precision) a valve operation controlled by software to deliver either a fixed volume or a fixed pressure output, wherein the electronic controlling does not require manual operation of valves, wherein simultaneous valve operation is triggered by sensing the pulsatile delivery of an output of accessory ventilators, wherein PEEP is achieved through use of CPAP rather than a resistance valve in an outflow track thus allowing precise control of PEEP.

Multiplier (M-) Mode-Sequential Operation with Accessory Ventilator Controlled I time and RR

In M-Mode, in a certain embodiments of the present disclosure, an accessory ventilator can deliver pulsatile airflow at fixed pressure (e.g., 50 cm H₂O, <60 cm H₂O, etc.), I time, and RR to the device (of the present disclosure) via a coupler. The number of patients that can be supported on the device can be a function of I time, I:E ratio and RR. Referring specifically to FIGS. 2A-2B, FIG. 4, and FIG. 5, an outlet manifold assembly 138 of multifunctional ventilation device 100 is illustrated. Within the multifunctional ventilation device 100, an air pressure (flow) sensor 112 can detect the pulsatile airflow from an existing ventilator 140 in order to synchronize operations of the multifunctional ventilation device 100 with that of the accessory ventilator 140. Each pulse of PIP from the ventilator 140 can trigger a sequential operation (e.g., patient #1-4) of the proportional master air valve 116, the proportional oxygen (O₂) valve 118, and the on/off operation of mini valves 120, 122, 124, 126 located at the outflow track of each patient. The proportional master air valve 116 can be set to provide the correct level of PIP for each patient. The PIP can be adjustable by increments of 1 cm H₂O (e.g., between 15 and 40 cm H₂O). The proportional oxygen valve 118 (which can open 50 ms after the proportional master air valve 116 valve opens and close 50 ms before the proportional master air valve 116 closes) can be adjusted to deliver the FiO₂ required by each patient. The FiO₂ can be adjustable by increments of 1% (e.g., between 21% and 100% O₂). The individualized PIP and O₂ is distributed through an outlet manifold 128 (e.g., a 4-way manifold, a patient manifold, a 3D printed manifold, etc.). The mini valves 120, 122, 124, 126, one on each limb of outlet manifold 128 (e.g., a 4-way outlet manifold), can open and close sequentially in sync with the proportional master air valve 116, in this way providing independent, sequential ventilation with individualized PIP and FiO₂ to the patients (e.g., patients 1-4) via patient outlets 144, 146, 148, 150 and couplers 158. In certain applications, air pressure sensors 130, 132, 134, 136 at the output side of the on/off mini valves 120, 122, 124, 126 can alarm if airflow is not detected for a certain time period (e.g., 20 seconds) or if the air pressure exceeds a preset limit due to a mechanical obstruction. PEEP can be emulated in multifunctional ventilation device 100 as described herein for S-Mode.

Certain methods of the present disclosure relate to implementing or using a multiplier mode. For example, one method includes the steps of: multiplying ventilator capacity through a sequential delivery of an output of an accessory ventilator multiple patients, wherein a sequential operation is triggered by sensing a pulsatile delivery of the accessory ventilator's output, wherein electronic operations are precisely controlled by software, wherein individualized FiO₂ is independently delivered to each patient, wherein a gas lens 152 is used to mix two (or more) gases to achieve a consistent gas mixture, wherein individualized PIP is independently delivered to each patient wherein use of a flow sensor (e.g., flow sensor 192) provides a feedback loop regulating the operation of the proportional air valve to assure delivery of the desired pressure.

### Pressurized Air (P-) Mode-Sequential Operation as a Standalone Pneumatic Ventilator

In P-Mode, in certain embodiments of the present disclosure, the device can serve as a standalone ventilator. A continuous source 142 of air pressure and O₂ feeds directly into the device, which controls RR, I time, PIP, FiO₂, and PEEP to provide ventilator support for a plurality of patient (e.g., 4 patients). RR and I time can be controlled through operation of the mini valves 120, 122, 124, 126. Independent ventilation and individualized PIP and FiO₂ can be achieved (e.g., as in M-Mode). The number of patients that can be simultaneously ventilated is a function of I time, I:E ratio and RR. Because of the relationship between RR and I time, in P-Mode a wider range of RR and I time can be achieved for a given number of patients. There is no difference in the architecture of the device operating in P-Mode or M-Mode (except that in P-Mode, the input air pressure sensor 168 is inactive).

Certain methods of the present disclosure relate to implementing or using a pressurized air mode. For example, one method includes the steps of: using a gas lens 152 to mix multiple (e.g., two) gases to achieve a consistent gas mixture; independently delivering individualized FiO₂ to each patient; independently delivering individualized PIP to each patient; and using a flow sensor to provide a feedback loop regulating an operation of a proportional air valve to assure delivery of the correct pressure.

### Cleaning Mode (C-Mode)—Sterilizing the Device

In C Mode, in a certain embodiments of the present disclosure, sterilizing gas can be sent through manifolds (e.g., manifolds 116, 128, etc.) of the multifunctional ventilation device 100 for a selected period of time. After a ventilator inlet port is connected to a sanitizing agent source, first the upper manifold can be sanitized and then the lower manifold can be sanitized (or vice versa). After sanitization, the manifolds can be flushed in the same manner (e.g., with sterile water). A start delay can permit the device to be placed in a sealed container before initiating a sterilization cycle.

### Description of a Preferred Embodiment

The following description describes an embodiment that integrates all functional modes, and is configured to be used with a patient load of 4. This description is exemplary in nature and not intended to be limited. Using quick connect couplers (e.g., coupler 156), a pressurized gas source (e.g., air) can be attached to the intake port 154 of a upper manifold 160 (e.g., PEEP manifold) and another pressurized gas source (e.g., oxygen) can be attached to an intake port 162 on a lower manifold 128 (e.g., lower patient manifold) of outlet manifold assembly 138 (e.g., second outlet manifold assembly). Gas (e.g., from 154) can feed upper manifold 160 (e.g., CPAP/PEEP manifold) of outlet manifold assembly 102 in all “Modes” of operation. In P-Mode, the majority of gas from the pressurized gas source can be diverted through a transfer valve 164 into the lower manifold 128 (e.g., lower patient manifold). In M-Mode and S-Modes, in addition to continuous gas from the pressurized gas source being delivered to the upper manifold 160 (e.g., CPAP/PEEP manifold) of outlet manifold assembly 102, an accessory ventilator unit attached to an intake port 166 on a lower manifold can deliver intermittent positive inspiratory pressurized gas into the lower manifold 128.

In all “Modes” of operation, gas directed into the upper manifold 160 (e.g., CPAP/PEEP manifold) of outlet manifold assembly 102 through 154 can first flow through a proportional master air valve 116. From proportional master air valve 116, the gas can then flow through the upper manifold 160 (e.g., CPAP/PEEP manifold) of outlet manifold assembly 102 to a plurality (e.g., four) separate output couplers (e.g., PEEP output couplers) (not shown). Gas can travel continuously and simultaneously to each output coupler, which can be attached to a patient respiration system (e.g., endotracheal tubing or a breathing mask) with flexible fluid conduits.

In M-Mode and S-Mode, gas from the accessory ventilator unit delivered through port 166 can flow past a pressure sensor 168 and through a proportional master air valve 116. After passing proportional master air valve 116, the gas can be mixed in the lower manifold 128 with the gas from intake port 162, and the gas mixture can be distributed to the on/off mini valves 120, 122, 124, 126. Gas from each on/off mini valve can flow past its respective pressure sensors (PS) 130, 132, 134, 136. From each PS, gas can flow to a patient output coupler 158. Each couple 158 can be attached to a patient respiration system with flexible fluid conduits. In P-Mode and M-Mode, gas can travel sequentially to on/off mini valves 120, 122, 124, 126; while in S-Mode, gas can travel simultaneously to on/off mini valves 120, 122, 124, 126. In S-Mode and M-Mode, pressure sensor 168 can synchronize the operation of proportional master air valve 116 and each on/off mini valve with the pulsatile flow of gas from the accessory ventilator unit. In P-Mode, the pressure sensor 168 can be inactive; the synchronized operation of proportional master air valve 116 and each on/off mini valve can be programmed in software (e.g., the “QV software”).

In P-Mode, gas from intake port 154 can be directed to both the upper manifold 160 (e.g., CPAP/PEEP manifold) of outlet manifold assembly 102 and, via the transfer valve 164, to the lower manifold 128. Gas can flow into the upper manifold 160 (e.g., CPAP/PEEP manifold) of outlet manifold assembly 102 (as it does in M-Mode and S-Mode).

Exhaled air from the patient can be sent through a nanofilter and sanitizing disinfectant before being vented to room air (not illustrated).

Such an embodiment can operate on a cyclic basis, one iteration of which follows a specified temporal order, including the following steps: (a) drive gases continually (ranging from “no flow” to “high flow”) to all patient air delivery systems on one manifold; (b) drive gases through (or “engage”) one ventilator branch while preventing flow through all other ventilator branches; (c) drive gases through a ventilator branch that previously had not been engaged, while preventing flow through all other ventilator branches including the branch that was just engaged; and repeat steps (b) and (c) until all ventilator branches have engaged exactly one (1) time (wherein step (a) always applies).

Each cycle corresponds to one (1) inspiration. Each patient exhalation will passively initiate after the driving gas is stopped, during which time the inspiratory cycle of the other patient(s) will initiate. Thus, total cycle time is dependent on the length and frequency of inspiration. The number of patients that can be ventilated at once is determined by the inspiratory time, inspiratory time to expiratory time ratio, and the respiratory rate. In the example in which the RR=15 (i.e. cycle time=4 sec), the I=1 sec, and the I:E=2, the indicated PIP and FiO₂ will be delivered to “patient 1” for one (1) second (during the time interval 0-1 second), after which “patient 1” exhalation will begin and continue for (2) seconds. The indicated PIP and FiO₂ will be delivered to “patient 2” for one (1) second (during the time interval 1-2 seconds), after which “patient 2” exhalation will begin and continue for two (2) seconds. The indicated PIP and FiO₂ will be delivered to “patient 3” for one (1) second (during the time interval 2-3 seconds), after which “patient 3” exhalation will begin and continue for two (2) seconds. The indicated PIP and FiO₂ will be delivered to “patient 4” for one (1) second (during the time interval 3-4 seconds), after which “patient 4” exhalation will begin and continue for 2 seconds. The sequence (e.g., patients 1-4) will repeat and the indicated PIP and FiO₂ will be delivered to each patient such that “patient 1” inspiration for one (1) second will occur during the time interval 4-5 seconds, after which “patient 1” exhalation will begin and continue for two (2) seconds, and so forth for patients 2-4.

These parameters are preferably set at a user interface (e.g., as in user interface 170 of FIG. 6). However, the scope of present disclosure is not so limited and could also cover any other system for parameter setting. Further parameters also include the fraction (i.e., mole fraction expressed as a percentage) of oxygen inspired, the peak inspiratory pressure, and the positive-end expiratory pressure. Embodiments of the present disclosure would incorporate these parameters as individualizable (i.e., different) for each patient air delivery system (e.g., fraction of oxygen inspired and peak inspiratory pressure in the presently described embodiment), or communal (i.e., homogenous) across patient air delivery systems (positive-end expiratory pressure, inspiratory time, respiratory rate, and the inspiratory time to expiratory time ratio in the presently described embodiment).

Pressure-sensing devices can be included at various places in the flow path, as seen diagrammatically in FIG. 4 and FIG. 5 and architecturally in FIGS. 2A-2B and FIG. 3. Such devices (e.g., sensors 130, 132, 134, 136, 168, PEEP manifold sensor 190, etc.) can detect flow, including any deviations or abnormalities, and issue warnings through visible and auditory alarms at the user interface 170. The present disclosure extends beyond the presently described embodiment to any configuration of sensing devices positioned to orchestrate the normal functioning of the aforementioned cycle, interacting with other components in various ways, and signaling users through various mechanisms. Additionally, should excessive pressure develop within the manifolds for any reason, manifold blowoff valve 176 or emergency blowoff valve 178 can open to dissipate that pressure.

Although the gases described herein relate often to oxygen and air, the invention is not so limited. For example, gases such as helium, nitrogen, or other suitable gases (and combination of gases) may be used herein. It should be understood that these gases are exemplary in nature and not intended to limit the embodiments contemplated herein.

Certain manifolds described herein can be made of a variety of materials. For example, the manifolds may be made from a material suitable for precision gas flow, including composite, plastic, or metallic materials. In certain embodiments, the first outlet manifold assembly and the second outlet manifold assembly are made from a composite material suitable for precision gas flow. In certain embodiments, the first outlet manifold assembly and the second outlet manifold assembly are made using an additive manufacturing process. The additive manufacturing process used can be a fused deposition modeling (FDM), a multi-material FDM, or another multi-material additive manufacturing technique. Such additive manufacturing processes can provide desired aspects, such as “ridges” or “corrugations” within the inner features (i.e., inner diameter wall). These ridges can be useful in mixing a combination of gases (e.g., air, oxygen, helium, etc.) within a manifold while travelling to, for example, patient delivery points or a patient air delivery system. It should be understood that these materials and processes are exemplary in nature and not intended to limit the embodiments contemplated herein.

### EQUIVALENTS

Although preferred embodiments of the invention have been described using specific terms, such description is for illustrative purposes only, and it is to be understood that changes and variations may be made without departing from the spirit or scope of the following claims.

### INCORPORATION BY REFERENCE

The entire contents of all patents, published patent applications, and other references cited herein are hereby expressly incorporated herein in their entireties by reference.

### Enumerated Embodiments

The following enumerated embodiments are provided, the numbering of which is not to be construed as designating levels of importance. In some aspects, the present disclosure is directed to the following non-limiting embodiments:

Embodiment 1 provides a multifunctional ventilation device comprising: an intake manifold assembly configured to receive a gas from at least one of a plurality of gas sources; a first outlet manifold assembly configured and adapted to be in fluidic connection with the intake manifold assembly, the first outlet manifold assembly including a first plurality of patient outlets, the first outlet manifold assembly being configured and adapted to deliver continuous airflow; a second outlet manifold assembly configured and adapted to be in fluidic connection with the intake manifold assembly, the second outlet manifold assembly including a second plurality of patient outlets, the second outlet manifold assembly being configured and adapted to deliver periodic, sequential or simultaneous airflow; a first proportional master air valve configured to provide precise airflow to the first outlet manifold assembly, the first proportional master air valve being in fluidic connection with at least the first outlet manifold assembly; a second proportional master air valve configured to provide precise airflow to the second outlet manifold assembly, the second proportional master air valve being in fluidic connection with at least the second outlet manifold assembly;

and a control system including: a plurality of valves including the first and second proportional master air valve; a plurality of sensors; and a processor configured to: control the plurality of valves, and measure data from the plurality of sensors; wherein the control system is programmed to selectively provide continuous, periodic, sequential or simultaneous delivery of the gas to a patient air delivery system; wherein each of the first plurality of patient outlets are fluidically equidistant from the first proportional master air valve; wherein each of the second plurality of patient outlets are fluidically equidistant from the second proportional master air valve.

Embodiment 2 provides the multifunctional ventilation device of embodiment 1, wherein the gas is selective from the group consisting of: air, oxygen, and a combination of air and oxygen; wherein the control system includes a single controller and is further programmed to provide continuous or simultaneous delivery of the gas in one or more modes including: an invasive mechanical ventilation (IMV) mode wherein a proportion of the combination of air and oxygen can be selected by a technician, or a positive end expiratory pressure (PEEP) emulation wherein a constant pre-settable air pressure is maintained.

Embodiment 3 provides the multifunctional ventilation device of any one of embodiments 1-2, wherein the first outlet manifold assembly includes a first plurality of outlet branches and the second outlet manifold assembly includes a second plurality of outlet branches, wherein each of the first and the second plurality of outlet branches defines a respective end, wherein each end is configured to be fluidically connected to a patient air delivery system.

Embodiment 4 provides the multifunctional ventilation device of any one of embodiments 1-3, wherein the first outlet manifold assembly and the second outlet manifold assembly are configured to direct gas in a plurality of directions to a plurality of patient delivery points.

Embodiment 5 provides the multifunctional ventilation device of any one of embodiments 1-4, wherein the second outlet manifold assembly and the control system are configured and adapted to indirectly connect to a standalone ventilator, wherein the multifunctional ventilation device is configured and adapted to operate in a splitter mode (S-Mode) such that a ventilator output is delivered to each of a plurality of patients.

Embodiment 6 provides the multifunctional ventilation device of any one of embodiments 1-5, wherein the multifunctional ventilation device is configured and adapted to couple to an existing ventilator and operate in a multiplier mode (M-Mode) such that positive inspiratory pressure (PIP) and fraction of inspired oxygen (FiO₂) are independently and sequentially delivered to each of a plurality of patients, wherein the multifunctional ventilation device is configured to adjust PIP and FiO₂ to individualized needs of each of a plurality of patients.

Embodiment 7 provides the multifunctional ventilation device of any one of embodiments 1-6, wherein the control system further comprises: an air pressure sensor configured to detect a pulsatile airflow from the existing ventilator; a proportional oxygen valve; and a plurality of mini valves disposed adjacent to the patient outlets, wherein the multifunctional ventilation device is configured and adapted to synchronize operations of the proportional master air valves, the proportional oxygen valve, or the plurality of mini valves with operations of the existing ventilator.

Embodiment 8 provides the multifunctional ventilation device of any one of embodiments 1-7, wherein the multifunctional ventilation device is configured and adapted to operate in a pressurized air mode (P-Mode) such that the multifunctional ventilation device functions as a standalone ventilator, wherein the multifunctional ventilation device is configured and adapted to receive a continuous source of air pressure and oxygen (O₂), such that positive inspiratory pressure (PIP) and fraction of inspired oxygen (FiO₂) are independently and sequentially delivered to each of a plurality of patients; wherein the multifunctional ventilation device is configured to adjust PIP and FiO₂ to individualized needs of each of a plurality of patients.

Embodiment 9 provides the multifunctional ventilation device of any one of embodiments 1-8, wherein the multifunctional ventilation device is configured to operate in a CPAP-Mode, such that pressure and supplied FiO₂ can be continuously adjusted and delivered equally to a plurality of patients.

Embodiment 10 provides the multifunctional ventilation device of any one of embodiments 1-9, wherein the control system is configured to execute a programmable self-cleaning function and sterilize at least one internal valve or at least one tubing with a disinfecting driving gas.

Embodiment 11 provides the multifunctional ventilation device of any one of embodiments 1-10, wherein the multifunctional ventilation device is configured to implement one or more functions selected from the group consisting of: S-Mode, M-Mode, P-Mode, CPAP-Mode, and a cleaning mode.

Embodiment 12 provides the multifunctional ventilation device of any one of embodiments 1-11, wherein each of the first outlet manifold assembly and the second outlet manifold assembly further comprise: a plurality of manifold branches configured and adapted to supply gas to up to four patient air delivery systems sequentially.

Embodiment 13 provides the multifunctional ventilation device of any one of embodiments 1-12, wherein each of the first outlet manifold assembly and the second outlet manifold assembly further comprise: a plurality of manifold branches configured and adapted to supply gas to a plurality of patient air delivery systems sequentially.

Embodiment 14 provides the multifunctional ventilation device of any one of embodiments 1-13, wherein each of the first outlet manifold assembly and the second outlet manifold assembly further comprise: a plurality of manifold branches configured and adapted to supply gas to up to four patient air delivery systems simultaneously.

Embodiment 15 provides the multifunctional ventilation device of any one of embodiments 1-14, wherein each of the first outlet manifold assembly and the second outlet manifold assembly further comprise: a plurality of manifold branches configured and adapted to supply gas to a plurality of patient air delivery systems.

Embodiment 16 provides the multifunctional ventilation device of any one of embodiments 1-15, wherein the control system includes a valve controller pre-programmed to operate the first and second master proportional valves in an operating cycle, the operating cycle including the steps of: (a) driving gas continually to a plurality of air delivery systems via the first outlet manifold assembly; (b) driving gases through one ventilator branch of a plurality of ventilator branches of the second outlet manifold assembly while preventing gas flow through all other ventilator branches; (c) driving gases through another ventilator branch of the plurality of ventilator branches, wherein the another ventilator branch has not been previously engaged, while preventing flow through the one ventilator branch and all other ventilator branches; and (d) repeating the driving of step (c) until each of the plurality of ventilator branches have engaged once.

Embodiment 17 provides the multifunctional ventilation device of any one of embodiments 1-16, wherein the control system is configure to operate by patient number. Embodiment 18 provides the multifunctional ventilation device of any one of embodiments 1-17, wherein the valve controller is pre-programmed to operate the first and second master proportional air valve in an operating cycle and to operate by patient number. Embodiment 19 provides the multifunctional ventilation device of any one of embodiments 1-18, wherein the control system further comprises: a pressure sensor capable of detecting pressure values outside of an expected range; and an alarm capable of signaling when the pressure sensor detects pressure values outside of the expected range.

Embodiment 20 provides the multifunctional ventilation device of any one of embodiments 1-19, further comprising: a plurality of pressure release valves configured and adapted to automatically depressurize a portion of the intake manifold assembly, the first outlet manifold assembly, or the second outlet manifold assembly in response to excess pressure buildup.

Embodiment 21 provides the multifunctional ventilation device of any one of embodiments 1-20, wherein the control system includes a plurality of one-way valves configured to prevent cross-contamination.

Embodiment 22 provides the multifunctional ventilation device of any one of embodiments 1-21, further comprising: a gas lens configured to uniformly mix the gas sources.

Embodiment 23 provides the multifunctional ventilation device of any one of embodiments 1-22, wherein the control system further comprises: an electronic user interface configured to receive and transmit ventilation parameters to the processor, wherein the processor is configured to determine parameter modifications, wherein the control system is configured and adapted to implement the parameter modifications.

Embodiment 24 provides the multifunctional ventilation device of any one of embodiments 1-23, wherein at least one of the plurality of gas sources is a separate ventilation system.

Embodiment 25 provides the multifunctional ventilation device of any one of embodiments 1-24, further comprising: a plurality of flowmeters, wherein the first outlet manifold assembly includes a first plurality of outlet branches; wherein the second outlet manifold assembly includes a second plurality of outlet branches; wherein each of the plurality of flowmeters is connected to a respective outlet branch.

Embodiment 26 provides the multifunctional ventilation device of any one of embodiments 1-25, wherein the control system is configured and adapted to measure and monitor pressure; wherein the control system is configured and adapted to detect deviations from a desired pressure and automatically adjust control parameters through safety feedback loops such that the desired pressure is achieved.

Embodiment 27 provides the multifunctional ventilation device of any one of embodiments 1-26, wherein the first outlet manifold assembly and the second outlet manifold assembly are made from a composite material suitable for precision gas flow.

Embodiment 28 provides the multifunctional ventilation device of any one of embodiments 1-27, wherein the first outlet manifold assembly and the second outlet manifold assembly are made using an additive manufacturing process.

Embodiment 29 provides the multifunctional ventilation device of any one of embodiments 1-28, wherein the additive manufacturing process used is a fused deposition modeling (FDM), a multi-material FDM, or another multi-material additive manufacturing technique.

1. A multifunctional ventilation device comprising:
an intake manifold assembly configured to receive a gas from at least one of a plurality of gas sources;
a first outlet manifold assembly configured and adapted to be in fluidic connection with the intake manifold assembly, the first outlet manifold assembly including a first plurality of patient outlets, the first outlet manifold assembly being configured and adapted to deliver continuous airflow;
a second outlet manifold assembly configured and adapted to be in fluidic connection with the intake manifold assembly, the second outlet manifold assembly including a second plurality of patient outlets, the second outlet manifold assembly being configured and adapted to deliver periodic, sequential or simultaneous airflow;
a first proportional master air valve configured to provide precise airflow to the first outlet manifold assembly, the first proportional master air valve being in fluidic connection with at least the first outlet manifold assembly;
a second proportional master air valve configured to provide precise airflow to the second outlet manifold assembly, the second proportional master air valve being in fluidic connection with at least the second outlet manifold assembly; and
a control system including:
a plurality of valves including the first and second proportional master air valve;
a plurality of sensors; and
a processor configured to:
control the plurality of valves, and
measure data from the plurality of sensors;
wherein the control system is programmed to selectively provide continuous, periodic, sequential or simultaneous delivery of the gas to a patient air delivery system;
wherein each of the first plurality of patient outlets are fluidically equidistant from the first proportional master air valve; and
wherein each of the second plurality of patient outlets are fluidically equidistant from the second proportional master air valve.

2. The multifunctional ventilation device of claim 1, wherein the gas is selected from the group consisting of: air, oxygen, and a combination of air and oxygen;
wherein the control system includes a single controller and is further programmed to provide continuous or simultaneous delivery of the gas in one or more modes; wherein said one or more modes are:
an invasive mechanical ventilation (IMV) mode wherein a proportion of the combination of air and oxygen can be selected by a technician, or
a positive end expiratory pressure (PEEP) emulation wherein a constant pre-settable air pressure is maintained.

3. The multifunctional ventilation device of claim 1, wherein the first outlet manifold assembly includes a first plurality of outlet branches and the second outlet manifold assembly includes a second plurality of outlet branches,
wherein each of the first and the second plurality of outlet branches defines a respective end, and
wherein each end is configured to be fluidically connected to a patient air delivery system.

4. The multifunctional ventilation device of claim 3, wherein the first outlet manifold assembly and the second outlet manifold assembly are configured to direct gas in a plurality of directions to a plurality of patient delivery points.

5. The multifunctional ventilation device of claim 1, wherein the second outlet manifold assembly and the control system are configured and adapted to indirectly connect to a standalone ventilator; and
wherein the multifunctional ventilation device is configured and adapted to operate in a splitter mode (S-Mode) such that a ventilator output is delivered to each of a plurality of patients.

6. The multifunctional ventilation device of claim 1, wherein the multifunctional ventilation device is configured and adapted to couple to an existing ventilator and operate in a multiplier mode (M-Mode) such that positive inspiratory pressure (PIP) and fraction of inspired oxygen (FiO₂) are independently and sequentially delivered to each of a plurality of patients, and
wherein the multifunctional ventilation device is configured to adjust PIP and FiO₂ to individualized needs of each of a plurality of patients.

7. The multifunctional ventilation device of claim 6, wherein the control system further comprises:
an air pressure sensor configured to detect a pulsatile airflow from the existing ventilator;
a proportional oxygen valve; and
a plurality of mini valves disposed adjacent to the patient outlets,
wherein the multifunctional ventilation device is configured and adapted to synchronize operations of the proportional master air valves, the proportional oxygen valve, or the plurality of mini valves with operations of the existing ventilator.

8. The multifunctional ventilation device of claim 1, wherein the multifunctional ventilation device is configured and adapted to operate in a pressurized air mode (P-Mode) such that the multifunctional ventilation device functions as a standalone ventilator,
wherein the multifunctional ventilation device is configured and adapted to receive a continuous source of air pressure and oxygen (O₂), such that positive inspiratory pressure (PIP) and fraction of inspired oxygen (FiO₂) are independently and sequentially delivered to each of a plurality of patients; and
wherein the multifunctional ventilation device is configured to adjust PIP and FiO₂ to individualized needs of each of a plurality of patients.

9. The multifunctional ventilation device of claim 1, wherein the multifunctional ventilation device is configured to operate in a CPAP-Mode, such that pressure and supplied FiO₂ can be continuously adjusted and delivered equally to a plurality of patients.

10. The multifunctional ventilation device of claim 1, wherein the control system is configured to execute a programmable self-cleaning function and sterilize at least one internal valve or at least one tubing with a disinfecting driving gas.

11. The multifunctional ventilation device of any of claims 1-10, wherein the multifunctional ventilation device is configured to implement one or more functions selected from the group consisting of: S-Mode, M-Mode, P-Mode, CPAP-Mode, and a cleaning mode.

12. The multifunctional ventilation device of claim 1, wherein each of the first outlet manifold assembly and the second outlet manifold assembly further comprise:
a plurality of manifold branches configured and adapted to supply gas to up to four patient air delivery systems sequentially.

13. The multifunctional ventilation device of claim 1, wherein each of the first outlet manifold assembly and the second outlet manifold assembly further comprise:
a plurality of manifold branches configured and adapted to supply gas to a plurality of patient air delivery systems sequentially.

14. The multifunctional ventilation device of claim 1, wherein each of the first outlet manifold assembly and the second outlet manifold assembly further comprise:
a plurality of manifold branches configured and adapted to supply gas to up to four patient air delivery systems simultaneously.

15. The multifunctional ventilation device of claim 1, wherein each of the first outlet manifold assembly and the second outlet manifold assembly further comprise:
a plurality of manifold branches configured and adapted to supply gas to a plurality of patient air delivery systems.

16. The multifunctional ventilation device of claim 1, wherein the control system includes a valve controller pre-programmed to operate the first and second master proportional valves in an operating cycle, the operating cycle including the steps of:
(a) driving gas continually to a plurality of air delivery systems via the first outlet manifold assembly;
(b) driving gases through one ventilator branch of a plurality of ventilator branches of the second outlet manifold assembly while preventing gas flow through all other ventilator branches;
(c) driving gases through another ventilator branch of the plurality of ventilator branches, wherein the another ventilator branch has not been previously engaged, while preventing flow through the one ventilator branch and all other ventilator branches; and
(d) repeating the driving of step (c) until each of the plurality of ventilator branches have engaged once.

17. The multifunctional ventilation device of claim 1, wherein the control system is configured to operate by patient number.

18. The multifunctional ventilation device of claim 1, wherein the valve controller is pre-programmed to operate the first and second master proportional air valve in an operating cycle and to operate by patient number.

19. The multifunctional ventilation device of claim 1, wherein the control system further comprises:
a pressure sensor capable of detecting pressure values outside of an expected range; and
an alarm capable of signaling when the pressure sensor detects pressure values outside of the expected range.

20. The multifunctional ventilation device of claim 1 further comprising:
a plurality of pressure release valves configured and adapted to automatically depressurize a portion of the intake manifold assembly, the first outlet manifold assembly, or the second outlet manifold assembly in response to excess pressure buildup.

21. The multifunctional ventilation device of claim 1, wherein the control system includes a plurality of one-way valves configured to prevent cross-contamination.

22. The multifunctional ventilation device of claim 1 further comprising:
a gas lens configured to uniformly mix the gas sources.

23. The multifunctional ventilation device of claim 1, the control system further comprising:
an electronic user interface configured to receive and transmit ventilation parameters to the processor,
wherein the processor is configured to determine parameter modifications, and
wherein the control system is configured and adapted to implement the parameter modifications

24. The multifunctional ventilation device of claim 1 wherein at least one of the plurality of gas sources is a separate ventilation system.

25. The multifunctional ventilation device of claim 1 further comprising:
a plurality of flowmeters,
wherein the first outlet manifold assembly includes a first plurality of outlet branches;
wherein the second outlet manifold assembly includes a second plurality of outlet branches; and
wherein each of the plurality of flowmeters is connected to a respective outlet branch.

26. The multifunctional ventilation device of claim 1 wherein the control system is configured and adapted to measure and monitor pressure; and
wherein the control system is configured and adapted to detect deviations from a desired pressure and automatically adjust control parameters through safety feedback loops such that the desired pressure is achieved.

27. The multifunctional ventilation device of claim 1, wherein the first outlet manifold assembly and the second outlet manifold assembly are made from a composite material suitable for precision gas flow.

28. The multifunctional ventilation device of claim 29, wherein the first outlet manifold assembly and the second outlet manifold assembly are made using an additive manufacturing process.

29. The multifunctional ventilation device of claim 29, wherein the additive manufacturing process used is a fused deposition modeling (FDM), a multi-material FDM, or another multi-material additive manufacturing technique.
